(19) United States
(12) Patent Application Publication
(10) Pub. No.: US 2026/0115722 A1
(43) Pub. Date: Apr. 30, 2026

(54) PRESSURE GENERATING DEVICE AND DETECTING SYSTEM INCLUDING THE SAME

(71) Applicant: Inti Taiwan, Inc., Zhubei City (TW)

(72) Inventors: Wei-Ming CHEN, Zhubei City (TW); I-Chiao HSIEH, Zhubei City (TW); Yung-Hsiang LIN, Zhubei City (TW); Chien-Hung CHEN, Zhubei City (TW)

(21) Appl. No.: 18/926,429

(22) Filed: Oct. 25, 2024

(51) Int. Cl. B01L 3/00

(52) U.S. Cl. CPC B01L 3/502761; B01L 2200/0647; B01L 2400/0487; B01L 2400/0633

(57) ABSTRACT

A pressure generating device includes a tank, a deformable membrane, a driving device, and a connection unit. The tank defines a chamber therein, and includes an opening and a communication port each of which is in fluid communication with the chamber. The deformable membrane is disposed to seal the opening. The driving device includes a driving shaft having an end which confronts the deformable membrane. The connection unit is configured to couple the driving shaft with the deformable membrane and to permit the deformable membrane to be driven by the driving shaft to transform between a flat state and a deformed state. A detecting system including the pressure generating device for detecting quality of a test sample is also provided.

### FIELD

The disclosure relates to a pressure generating device, and more particularly to a pressure generating device and a detecting system including the same for detecting quality of an oocyte or an embryo.

### BACKGROUND

In vitro fertilization (IVF) is an option for achieving pregnancy for couples suffering from infertility. In an IVF cycle, after stimulation of the ovaries of the mother using follicle stimulating hormone, several oocytes can be harvested from the ovaries and fertilized in vitro, and then one or more of the fertilized oocytes may be transferred back to the mother. In some cases, the fertilized oocytes may be further developed into embryos in vitro, and then one or more of the embryos are transferred back to the mother. To increase the live-birth rate for an IVF cycle, there is a need to select the fertilized oocytes or the embryos with better quality to be transferred back to the mother.

### SUMMARY

Therefore, an object of the disclosure is to provide a pressure generating device and a detecting system including the pressure generating device that can be used for determining quality of an oocyte or an embryo.

According to a first aspect of the disclosure, a pressure generating device includes a tank, a deformable membrane, a driving device and a connection unit. The tank has a chamber therein, and includes an opening and a communication port each of which is in fluid communication with the chamber. The deformable membrane is disposed to seal the opening, and is deformable between a flat state and a deformed state. The driving device is mounted to the tank and includes a motor body and a driving shaft. The driving shaft has a first shaft end that is disposed to confront the deformable membrane, and is driven by the motor body to move between a distal position, where the first shaft end is distal from the motor body, and a proximate position, where the first shaft end is proximate to the motor body. The connection unit is configured to couple the second shaft end with the deformable membrane and to permit the deformable membrane to be driven by the driving shaft to transform between the flat state, where the first shaft end is in one of the distal portion and the proximate portion, and the deformed state, where the first shaft end is in the other one of the distal position and the proximate position, such that a predetermined pressure is generated through the communication port when the deformable membrane is moved from one of the flat state and the deformed state to the other one of the flat state and the deformed state.

According to a second aspect of the disclosure, a detecting system is provided for detecting quality of a test sample including an oocyte or an embryo. The detecting system includes the pressure generating device, a micropipette for sucking the test sample, and a connecting tube disposed to connect the communication port of the tank to the micropipette for applying the predetermined pressure to the test sample.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating a pressure generating device according to a first embodiment of the disclosure.

FIG. 2 is a schematic view of the pressure generating device similar to FIG. 1 but in a pressure increasing state.

FIG. 3 is a schematic view illustrating a pressure generating device according to a second embodiment of the disclosure.

FIG. 4 is schematic view of the pressure generating device which is similar to FIG. 3 but in a pressure decreasing state.

FIG. 5 is a perspective view illustrating a pressure generating device according to a third embodiment of the disclosure.

FIG. 6 is an exploded perspective view illustrating some elements of the pressure generating device shown in FIG. 5.

FIG. 7 is an exploded perspective view illustrating a side casing of a tank, a deformable membrane, and a connection unit in the pressure generating device shown in FIG. 5.

FIG. 8 is an exploded perspective view illustrating the side casing of the tank, the deformable membrane, and the connection unit which is similar to FIG. 7 but in an opposite direction.

FIG. 9 is an exploded perspective view illustrating a drive device, a connection sleeve of the connection unit, and a flag piece in the pressure generating device shown in FIG. 5.

FIG. 10 is a schematic view illustrating a major part of a detecting system according to an embodiment of the disclosure.

FIG. 11 is a schematic view illustrating a microscope of the detecting system according to an embodiment of the disclosure.

FIG. 12 is a graph illustrating a testing process for determining quality of a test sample in accordance with some embodiments.

FIGS. 13 and 14 are two microscope images respectively illustrating two states of an oocyte before and after a predetermined negative pressure is provided by the pressure generating device of the detecting system;

FIG. 15 is a graph illustrating another testing process for determining quality of a test sample in accordance with some embodiments.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

It should be noted that the drawings, which are for illustrative purposes only, are not drawn to scale, and are not intended to represent the actual sizes or actual relative sizes of the components of the device or the system in this disclosure.

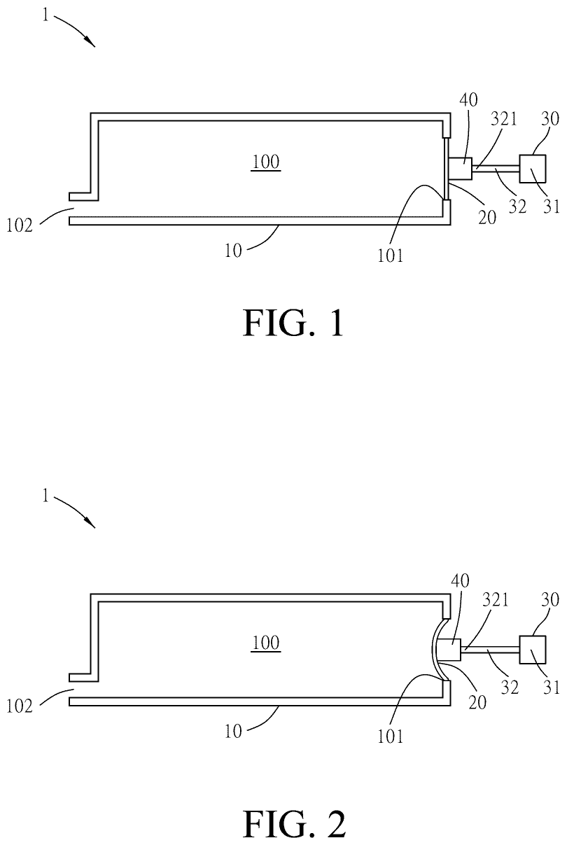

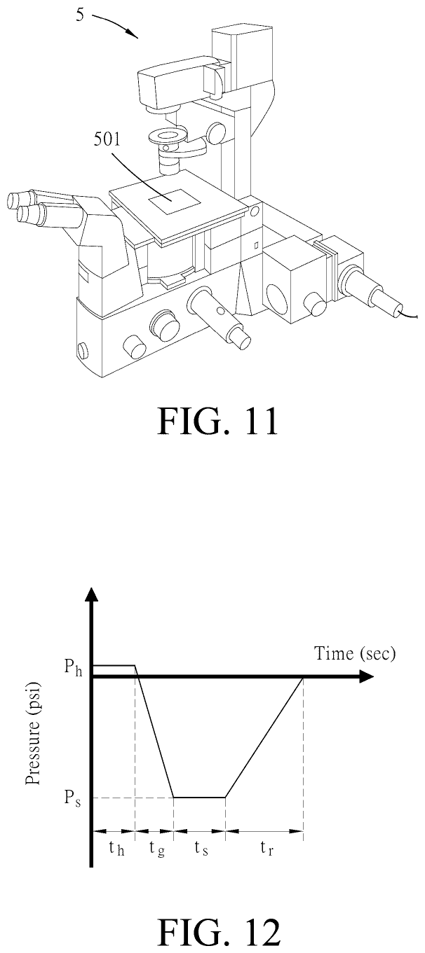

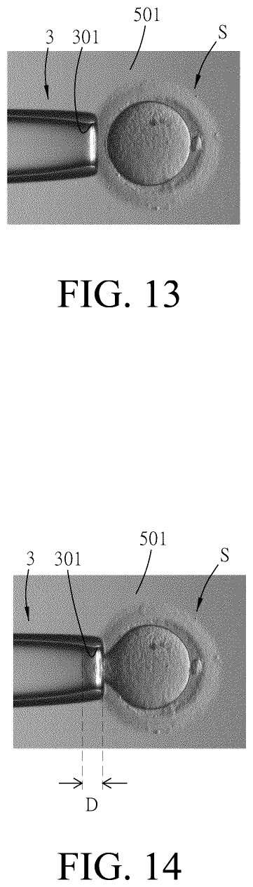

FIGS. 1 and 2 each shows a pressure generating device 1 according to a first embodiment of the disclosure. The pressure generating device 1 is used for generating a pressure to a test sample (S) through a micropipette 3 (see FIGS. 13 and 14). The test sample (S) may be an oocyte (e.g., a mammalian oocyte which may be fertilized or unfertilized) or an embryo (e.g., a mammalian embryo). Based on an aspiration depth (D) of the test sample (S) (see FIG. 14) and other factors (such as morphologies observed using a microscope 5 shown in FIG. 11), the quality of the test sample can be determined.

The pressure generating device 1 includes a tank 10, a deformable membrane 20, a driving device 30, and a connection unit 40.

The tank 10 has a chamber 100 therein, and includes an opening 101 and a communication port 102 each of which is in fluid communication with the chamber 100. The deformable membrane 20 is disposed to seal the opening 101, and is deformable between a flat state and a deformed state. The driving device 30 may be mounted to the tank 10 through any possible elements, and includes a motor body 31 and a driving shaft 32. The driving shaft 32 has a first shaft end 321 that is disposed to confront the deformable membrane 20. The driving shaft 32 is driven by the motor body 31 to move between a distal position, where the first shaft end 321 is distal from the motor body 31, and a proximate position, where the first shaft end 321 is proximate to the motor body 31. The connection unit 40 is configured to couple the first shaft end 321 with the deformable membrane 20 and to permit the deformable membrane 20 to be driven by the driving shaft 32 to transform between the flat state and the deformed state. In the flat state, the first shaft end 321 is in one of the distal portion and the proximate portion, and in the deformed state, the first shaft end 321 is in the other one of the distal position and the proximate position, such that a predetermined pressure is generated through the communication port 102 when the deformable membrane 20 is moved from one of the flat state and the deformed state to the other one of the flat state and the deformed state.

In the first embodiment, in the flat state, as shown in FIG. 1, the first shaft end 321 is in the proximate position; while in the deformed state, as shown in FIG. 2, the first shaft end 321 is in the distal position. As such, a predetermined pressure is generated through the communication port 102 when the deformable membrane 20 is moved from one of the flat state and the deformed state to the other one of the flat state and the deformed state.

In the first embodiment, in response to movement of the driving shaft 32 from the proximate position (see FIG. 1) to the distal position (see FIG. 2), the deformable membrane 20 is forced by the connection unit 40 to be deformed inwardly so as to convert the deformable membrane 20 from the flat state into the deformed state. With the deformation of the deformable membrane 20, the chamber 100 is converted into a pressure increasing state. In this case, once the deformable membrane 20 returns back to the flat state from the deformed state, the predetermined pressure can be generated from the communication port 102.

In some embodiments, the tank 10 may be made of metals, alloys, plastics, other suitable air-impermeable rigid materials, or combinations thereof. In some embodiments, the deformable membrane 20 may be made of silicone, polymer, fabric, any suitable air-impermeable flexible materials, or combinations thereof.

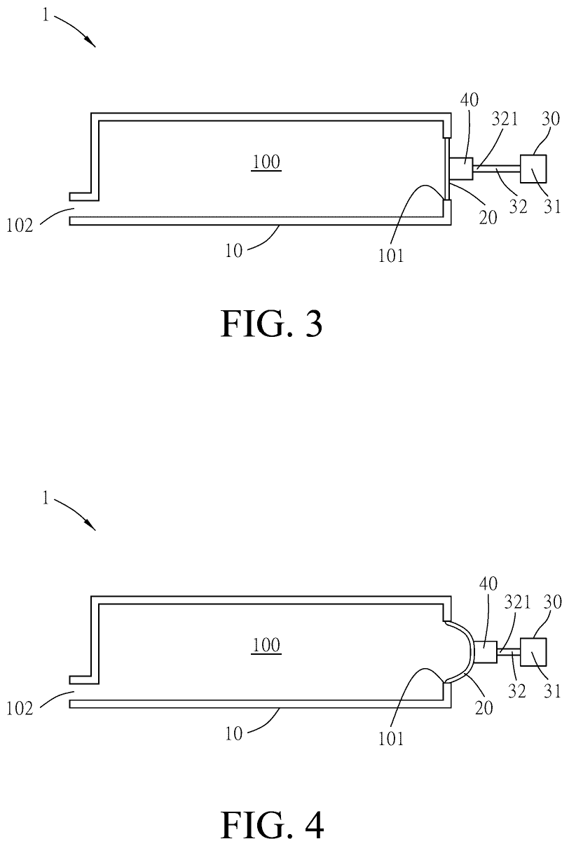

FIGS. 3 and 4 illustrate a pressure generating device 1 according to a second embodiment of the disclosure. The second embodiment is similar to the first embodiment, except that in the second embodiment, the deformable membrane 20 is deformed outwardly when in the deformed state. To be specific, in the second embodiment, the connection unit 40 is secured to the deformable membrane 20. When the driving shaft 32 is in the distal position, the deformable membrane 20 can be kept at the flat state. In response to movement of the driving shaft 32 to the proximate position (see FIG. 4) from the distal position (see FIG. 3), the deformable membrane 20 is pulled by the connection unit 40 to be deformed outwardly so as to convert the deformable membrane 20 from the flat state into the deformed state. With the deformation of the deformable membrane 20, the chamber 100 is converted into a pressure reducing state, and thus the predetermined pressure can be generated from the communication port 102.

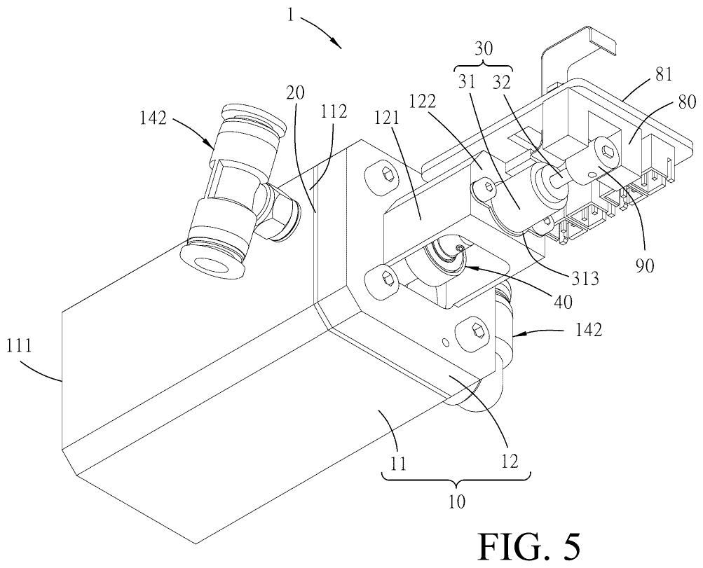

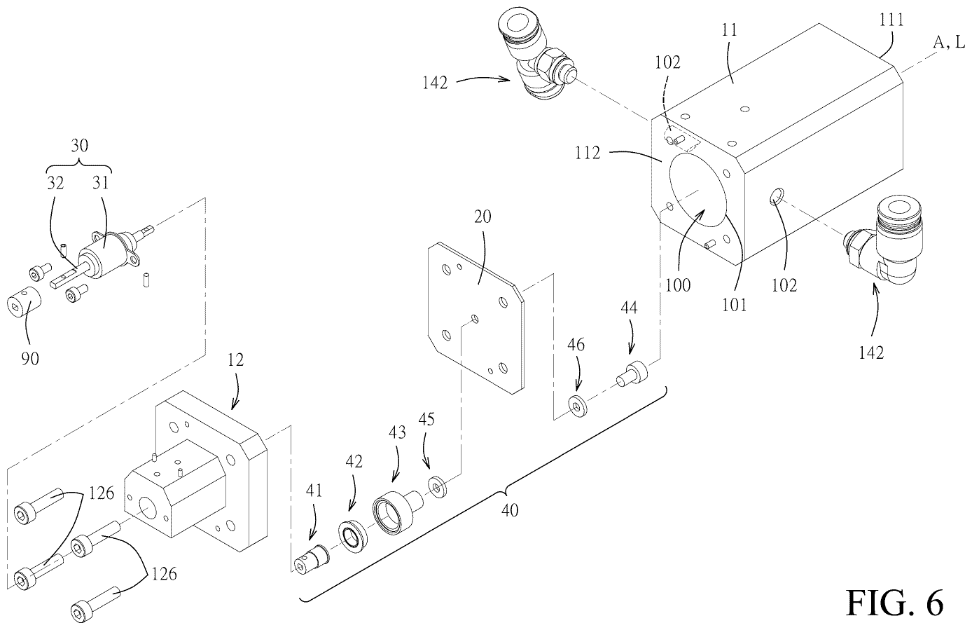

FIGS. 5 to 9 illustrate a pressure generating device 1 according to a third embodiment of the disclosure. The third embodiment is similar to the first or second embodiment, except that in the third embodiment, the tank 10, the deformable membrane 20, the driving device 30, and the connection unit 40 are shown in detail.

As shown in FIGS. 5 and 6, the tank 10 includes a main casing 11 and a side casing 12. The main casing 11 has the chamber 100 therein, and includes the opening 101 and the communication port 102. The side casing 12 is detachably mounted to the main casing 11 such that the deformable membrane 20 is secured between the main casing 11 and the side casing 12 to thereby seal the opening 101.

In some embodiments, the main casing 11 extends along a longitudinal axis (L) to terminate at a closed end 111 and a connection end 112 formed with the opening 101. In some embodiments, the main casing 11 is formed with two of the communication ports 102.

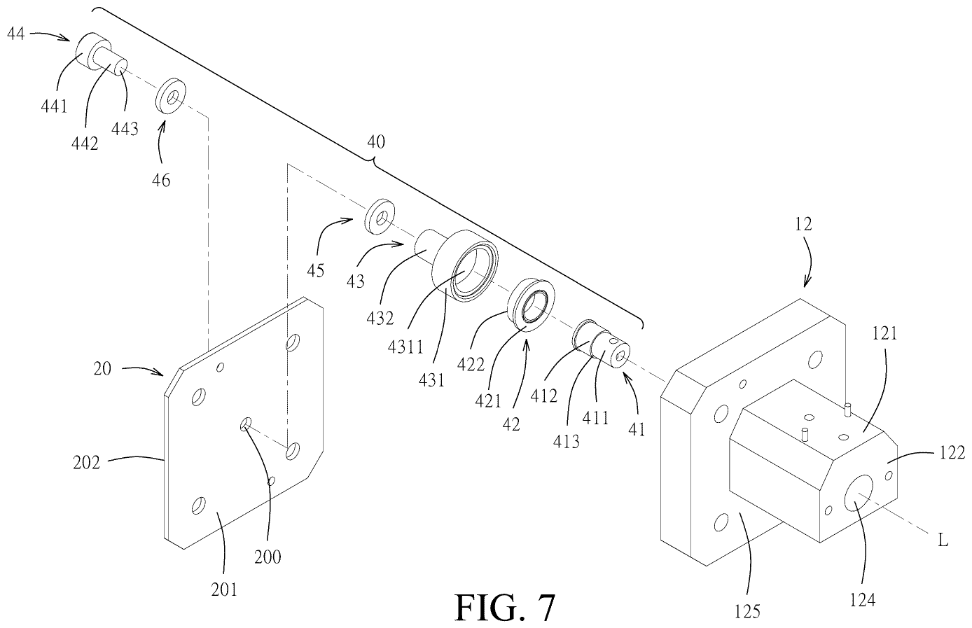

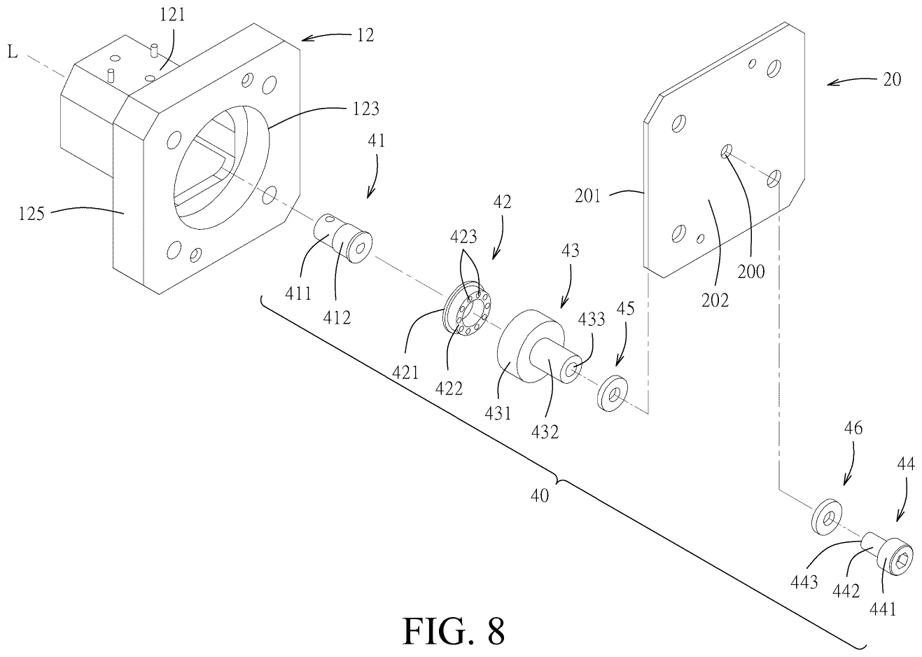

In some embodiments, referring to FIGS. 7 and 8, the side casing 12 includes a side casing body 121 and a flange body 125. The side casing body 121 has a distal end 122 and a proximal end 123 opposite to the distal end 122, and is formed with a passage 124 extending from the distal end 122 to the proximal end 123. The flange body 125 extends radially and outwardly from the proximate end 123 of the side casing body 121, and is detachably mounted to the main casing 11. In some embodiments, the deformable membrane 20 is kept at the flat state by a retaining force provided between the main casing 11 and the side casing 12.

In some embodiments, the tank 10 further includes two connection stems 142. Each of the connection stems 142 has an end that is inserted into a respective one of the communication ports 102. As such, the chamber 100 can be in fluid communication with a connection tube 4 (see FIGS. 1 to 4 and 10) through one of the connection stems 142. Each of the connection stems 142 may have a valve (not shown) mounted thereon, and the valve is switchable between a first state, where the one of the connection stems 142 is in an open state to permit the chamber 100 to be in fluid communication with the connection tube 4, and a second state, where the one of the connection stems 142 is in a closed state to block the communication between the chamber 100 and the connection tube 4.

In some embodiments, a plurality of fasteners 126 are provided to pass through the flange body 125 and the deformable membrane 20, and extend into the connection end 112 of the main casing 11 so as to fasten the side casing 12 to the main casing 11.

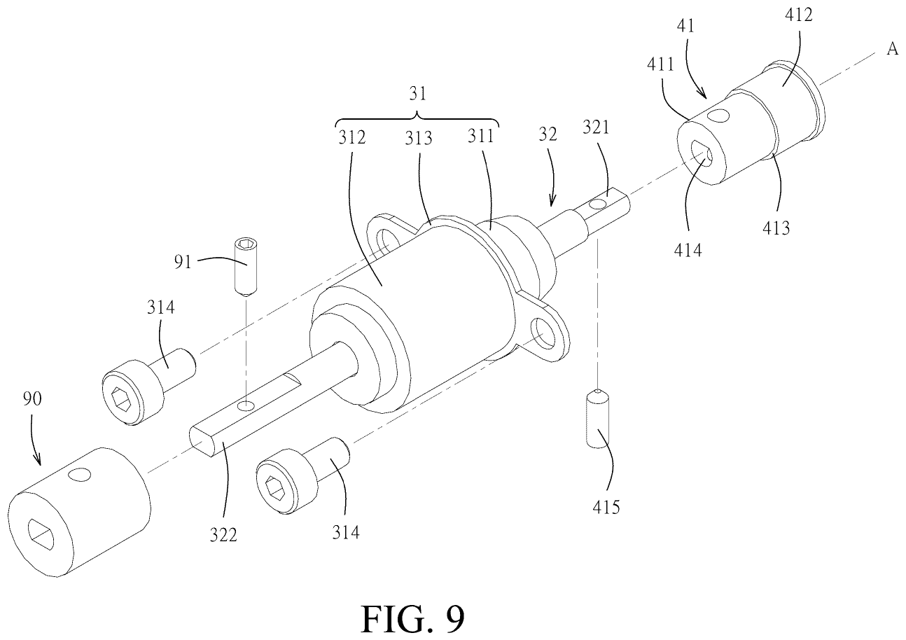

As shown in FIGS. 5 and 9, the driving device 30 is mounted to the tank 10. In some embodiments, the motor body 31 is immovably mounted to the side casing 12, and the driving shaft 32 extends along a shaft axis (A) through the motor body 31, and is driven by the motor body 31 to move toward or away from the deformable membrane 20 along the shaft axis (A) so as to transform the deformable membrane 20 between the flat state and the deformed state. In some embodiments, the shaft axis (A) may be in alignment with the longitudinal axis (L). In some embodiments, the driving device 30 is a linear motor, and the driving shaft 32, when being driven to move along the shaft axis (A), is not rotatable about the shaft axis (A). In some other embodiments, the driving device 30 is a stepper motor, and the driving shaft 32, when being driven to move along the shaft axis (A), rotates about the shaft axis (A).

In some embodiments, referring to FIG. 9, the motor body 31 has an inner portion 311, an outer portion 312 and a flange portion 313. The inner portion 311 is received in the passage 124 of the side casing body 121. The outer portion 312 extends from the inner portion 311 in a direction away from the main casing 11, and is disposed outside the side casing body 121. The flange portion 313 extends radially from the outer portion 312, and is detachably secured to the distal end 122 of the side casing body 121 by fasteners 314.

In some embodiments, the driving shaft 32 further has a second shaft end 322 which is opposite to the first shaft end 321 along the shaft axis (A). The first and second shaft ends 321, 322 are located at two opposite sides of the motor body 31.

As shown in FIGS. 7 and 8, in some embodiments, the connection unit 40 includes a connection sleeve 41, a bearing sleeve 42, and a bearing cap 43.

The connection sleeve 41 is coupled on the first shaft end 321 of the driving shaft 32 so as to permit the connection sleeve 41 to move with the driving shaft 32 along the shaft axis (A). The connection sleeve 41 has a first sleeve portion 411, a second sleeve portion 412, and a shoulder surface 413. The second sleeve portion 412 has an outer dimension that is larger than an outer dimension of the first sleeve portion 411. The shoulder surface 413 is located between the first sleeve portion 411 and the second sleeve portion 412. In some embodiments, as shown in FIG. 9, the connection sleeve 41 is formed with a through bore 414, and the first shaft end 321 is non-rotatably secured in the through bore 414 by a fastener 415.

The bearing sleeve 42 is fittingly sleeved on the second sleeve portion 412 so as to permit the bearing sleeve 42 to move with the connection sleeve 41 along the shaft axis (A), and is disposed between the connection sleeve 41 and the bearing cap 43. The bearing sleeve 42 has a first abutment end 421 and a second abutment end 422 opposite to the first abutment end 421. In some embodiments, a plurality of bearing balls 423 are formed in the second abutment end 422 and are angularly displaced from each other about the shaft axis (A).

The bearing cap 43 has an engaging portion 431 and a mounted portion 432. The engaging portion 431 is formed with a recess 4311 that is configured to engage with the second abutment end 422 so as to permit the bearing cap 43 to move with the bearing sleeve 42 along the shaft axis (A). The mounted portion 432 is opposite to the engaging portion 431 in the shaft axis (A), and is secured to a first surface 201 of the deformable membrane 20 so as to transmit a force from the driving shaft 32 to the deformable membrane 20.

In some embodiments, the deformable membrane 20 is formed with a through hole 200, and the connection unit 40 further includes a fastener 44. The fastener 44 has an enlarged head 441 and a fastening rod 442. The enlarged head 441 is disposed on a second surface 202 of the deformable membrane 20 opposite to the first surface 201. The fastening rod 442 extends from the enlarged head 441 through the through hole 200 of the deformable membrane 20 to terminate at a rod end 443. The rod end 443 is fittingly engaged within a hole 433 formed in the mounted portion 432 of the bearing cap 43 so as to permit the deformable membrane 20 to be fastened to the bearing cap 43 through the fastener 44.

In some embodiments, the connection unit 40 further includes a first washer 45 and a second washer 46. The first washer 45 is disposed between the mounted portion 432 and the first surface 201 of the deformable membrane 20. The second washer 46 is disposed between the enlarged head 441 and the second surface 202 of the deformable membrane 20. In some embodiments, in order to improve transmission of a torque from the driving shaft 32 to the deformable membrane 20, each of the first and second washers 45, 46 is made of a deformable material. Examples for the deformable material may be similar to the materials suitable for forming the deformable membrane 20, and thus the details thereof are omitted for the sake of brevity.

In some embodiments, with reference to FIGS. 1, 2 and 5 to 9, the deformable membrane 20 is deformed inwardly in the deformed state. In other words, in response to movement of the driving shaft 32 from the proximate position to the distal position, the bearing cap 43 is forced by the first shaft end 321 (through the connection sleeve 41 and the bearing sleeve 42) to move toward the chamber 100 and is brought into pressing engagement with the deformable membrane 20 so as to force the deformable membrane 20 to deform into the deformed state against the retaining force provided between the main casing 11 and the side casing 12. In response to movement of the driving shaft 32 from the distal position (see FIG. 2) to the proximate position (see FIG. 1), the bearing cap 43 is brought to move away from the chamber 100 so as to permit the deformable membrane 20 to return back to the flat state by the retaining force, thereby allowing the communication port 102 (or one of the communication ports 102 shown in FIG. 6) to generate the predetermined pressure (e.g., a predetermined negative pressure).

In some other embodiments, with reference to FIGS. 3 to 9, the deformable membrane 20 is deformed outwardly in the deformed state. In this case, the driving device 30 is a linear motor, and the connection sleeve 41, the bearing sleeve 42 and the bearing cap 43 are secured to each other. In some embodiments, every two adjacent ones of the connection sleeve 41, the bearing sleeve 42 and the bearing cap 43 are fittingly engaged with each other. In some other embodiments, an adhesive agent may be additionally applied between every two adjacent ones of the connection sleeve 41, the bearing sleeve 42 and the bearing cap 43. To be specific, in response to movement of the driving shaft 32 from the distal position (see FIG. 3) to the proximate position (see FIG. 4), the deformable membrane 20 is pulled by the connection unit 40 so as to deform outwardly into the deformed state against the retaining force between the main casing 11 and the side casing 12, thereby generating the predetermined pressure (e.g., a predetermined negative pressure) from the communication port 102 or one of the communication ports 102 shown in FIG. 6. In response to movement of the driving shaft 32 from the proximate position to the distal position, the connection unit 40 moves toward the chamber 100 so as to permit the deformable membrane 20 to return back to the flat state by the retaining force.

In some embodiments, the deformable membrane 20 has a hardness ranging from 0 Shore A to 100 Shore A, e.g., from 0 Shore A to 10 Shore A, from 5 Shore A to 15 Shore A, from 10 shore A to 20 Shore A, from 15 Shore A to 25 Shore A, from 20 Shore A to 30 Shore A, from 25 Shore A to 35 Shore A, from 30 Shore A to 40 Shore A, from 35 Shore A to 45 Shore A, from 40 Shore A to 50 Shore A, from 45 Shore A to 55 Shore A, from 50 Shore A to 60 Shore A, from 55 Shore A to 65 Shore A, from 60 Shore A to 70 Shore A, from 65 Shore A to 75 Shore A, from 70 Shore A to 80 Shore A, from 75 Shore A to 85 Shore A, from 80 Shore A to 90 Shore A, from 85 Shore A to 95 Shore A, or from 90 Shore A to 100 Shore A. The driving device 30 is configured to provide a thrust which ranges from 100 g to 1000 g, e.g., from 100 g to 200 g, from 150 g to 250 g, from 200 g to 300 g, from 250 g to 350 g, from 300 g to 400 g, from 350 g to 450 g, from 400 g to 500 g, from 450 g to 550 g, from 500 g to 600 g, from 550 g to 650 g, from 600 g to 700 g, from 650 g to 750 g, from 700 g to 800 g, from 750 g to 850 g, from 800 g to 900 g, from 850 g to 950 g, or from 900 g to 1000 g. A stroke of the driving device 30 ranges from 3 mm to 50 mm, e.g., from 3 mm to 15 mm, from 9 mm to 21 mm, from 15 mm to 27 mm, from 21 mm to 33 mm, from 27 mm to 39 mm, from 33 mm to 45 mm, or from 39 mm to 50 mm.

The stroke and the thrust of the driving device 30 are determined based on a required degree of deformation of the deformable membrane 20 and the hardness of the deformable membrane 20, respectively. When the stroke required for the driving device 30 is relatively long and the hardness of the deformable membrane 20 is relatively small, operation of the driving device 30 is relatively easier (i.e., the thrust required for the driving device 30 is relatively small). When the hardness of the deformable membrane 20 becomes larger, the thrust required for the driving device 30 will be increased if the stroke required for the driving device 30 is not reduced.

In some embodiments, a volume of the main casing 11 ranges from 1 cm³ to 50 cm³, e.g., from 1 cm³ to 10 cm³, from 5 cm³ to 15 cm³, from 10 cm³ to 20 cm³, from 15 cm³ to 25 cm³, from 20 cm³ to 30 cm³, from 25 cm³ to 35 cm³, from 30 cm³ to 40 cm³, from 35 cm³ to 45 cm³, or from 40 cm³ to 50 cm³.

In some embodiment, the pressure generating device 1 further includes a photo sensor 80 which is immovable relative to the tank 10 so as to permit the photo sensor 80 to detect a displacement of the driving shaft 32. In some embodiments, a circuit board 81 is mounted to the side casing body 121, and the photo sensor 80 is mounted on the circuit board 81 and is electrically connected to a circuit of the circuit board 81.

In some embodiment, referring to FIGS. 5 and 9, the pressure generating device 1 further includes a flag piece 90 which is mounted on the second shaft end 322 of the driving shaft 32 so as to permit the flag piece 90 to move with the driving shaft 32, and which has a plurality of detectable positions to be detected by the photo sensor 80, thereby determining the displacement of the driving shaft 32. In some embodiments, the flag piece 90 is mounted to the second shaft end 322 by a fastener 91.

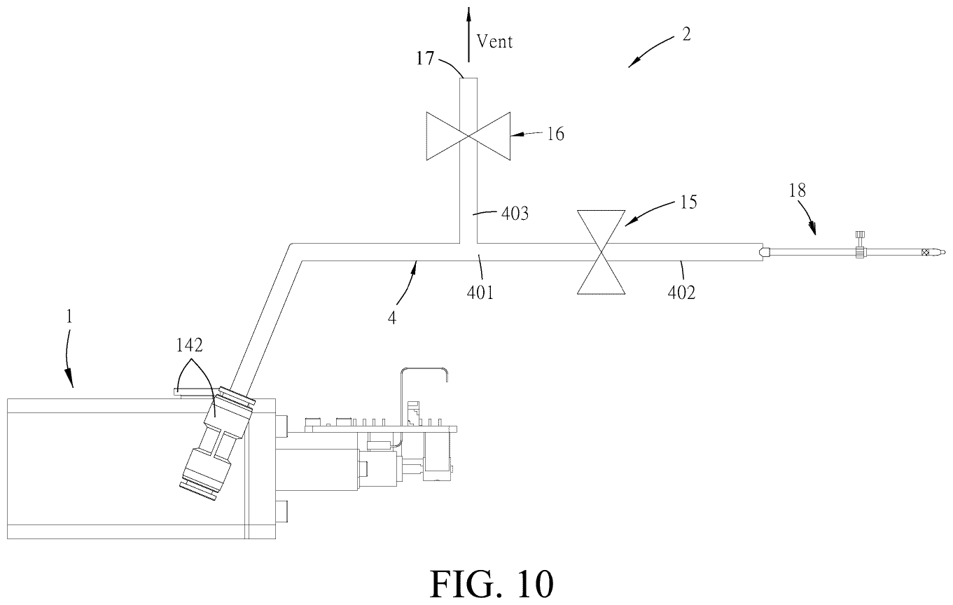

FIG. 10 illustrates a major part of a detecting system 2 for detecting quality of the test sample (S, see FIGS. 13 and 14) according to an embodiment of the disclosure. The detecting system 2 includes the pressure generating device 1 (in which one of the connection stems 142 is in the open state, the other one of the connection stems 142 is in the closed state), the micropipette 3 (see FIGS. 13 and 14) for sucking the test sample (S), and the connecting tube 4 disposed to connect one of the communication ports 102 of the tank 10 (through the one of the connection stems 142) to the micropipette 3 for applying the predetermined pressure (e.g., a predetermined negative pressure) from the pressure generating device 1 to the test sample (S). In some other embodiments, the detecting system 2 may include the pressure generating device 1 (in which both the connection stems 142 are in the open state), two of the micropipettes 3 (only one of which is shown in FIGS. 13 and 14) and two of the connecting tubes 4 (only one of which is shown in FIG. 10) so that the predetermined pressure from the pressure generating device 1 can be applied to two test samples(S) (only one of which is shown) through the connection stems 142, respectively. In the followings, each of the communication port 102, the connection stem 142, the micropipette 3, the connection tube 4 and so on is described in a singular form.

In some embodiments, an inner diameter of the micropipette 3 ranges from about 10 microns to about 100 microns (e.g., about 40 microns to about 70 microns).

In some embodiments, by the pressure generating device 1, a pressure ranging from about 0.5 psi to about −0.5 psi is generated at a suction port 301 of the micropipette 3. For example, the pressure generated at the suction port 301 of the micropipette 3 may varied from about −0.03 psi to about −0.5 psi, from about 0.03 psi to about −0.5 psi, from about 0.03 psi to about 0.5 psi, or from about −0.03 psi to about 0.5 psi.

In some embodiments, referring to FIGS. 10, 11, 13 and 14, the detecting system 2 further includes a microscope 5 and an injection holder 18. The microscope 5 is used for monitoring the test sample (S) and the suction port 301 of the micropipette 3. To be specific, the test sample (S) is placed on a dish 501 of the microscope 5 and the injection holder 18 is connected between the connecting tube 4 and the micropipette 3. In operation, the suction port 301 of the micropipette 3 is retained by the injection holder 18 to be disposed on the dish 501 of the microscope 5, so as to facilitate the test sample (S) on the dish 501 to be retained and/or sucked by the suction port 301 of the micropipette 3.

In some embodiments, the connecting tube 4 includes a first tube segment 401 connected to the communication port 102 of the tank 10, and a second tube segment 402 connected to the micropipette 3 through the injection holder 18. The first and second tube segments 401, 402 are connected to each other through an adjusting valve 15 for adjusting the pressure generated at the suction port 301 of the micropipette 3. In some embodiments, the adjusting valve 15 is a solenoid valve.

In some embodiments, the detecting system 2 further includes a branch tube 403 which is connected to the first tube segment 401, and which has the venting port 17 opposite to the first tube segment 401. In addition, a venting valve 16 is coupled to the branch tube 403 to control a fluid communication between the chamber 100 and the venting port 17. To be specific, when the venting valve 16 is opened, the venting port 17 is in fluid communication with the chamber 100, and when the venting valve 16 is fully closed, the venting port 17 is prevented from being in fluid communication with the chamber 100. Therefore, when the venting valve 16 is opened, the pressure inside the chamber 100 is permitted to return to an atmospheric pressure. In some embodiments, the venting valve 16 is a solenoid valve.

FIG. 12 is a graph illustrating a testing process for determining the quality of the test sample (S) in accordance with some embodiments. The testing process shown in FIG. 12 is described with reference to FIGS. 10, 13 and 14. During the testing process illustrated in FIG. 12, the adjusting valve 15 is always opened.

Referring to FIGS. 10 and 12, in the beginning, the venting valve 16 is fully closed, and the pressure inside the chamber 100 is adjusted to and kept at an initial pressure (e.g., a slightly positive or negative pressure), and the position of the actuating arm 40 is also moved to permit the deformable membrane 20 to be kept at a home state. For example, in the case that the deformable membrane 20 is deformed inwardly (e.g., the embodiment shown in FIGS. 1 and 2), the home state of the deformable membrane 20 is the deformed state (see FIG. 2); while in the case that the deformable membrane 20 is deformed outwardly (e.g., the embodiment shown in FIGS. 3 and 4), the home state of the deformable membrane 20 is the flat state (see FIG. 3). Due to the initial pressure of the chamber 100, as shown in FIG. 12, a slightly positive pressure (Pₕ) (e.g., about 0.01±10% psi) is generated at the suction port 301 of the micropipette 3 and kept for a time period (tₕ), and thus the test sample (S) is retained by the suction port 301 of the micropipette 3 and is prevented from being sucked into the micropipette 3 (see FIG. 13). Then, by actuating the driving device 30 to cause transformation of the deformable membrane 20, a pressure from the communication port 102 is continuously reduced for a time period (t_{g}) (e.g., 2±10% seconds) so as to reach the predetermined pressure (e.g., a predetermined negative pressure), and meanwhile, a suction pressure generated at the suction port 301 of the micropipette 3 is gradually decreased to a stress pressure (Pₛ) (e.g., −0.1±10% psi). The stress pressure (Pₛ) lasts for a time period (tₛ) (e.g., 1±10% second) and at this stage, as shown in a microscope image in FIG. 14, an aspiration depth (D) of the test sample (S) is determined using a computer program (not shown). Finally, the driving device 30 is further actuated to continuously increase the pressure inside the chamber 100 for a time period (tᵣ), thereby returning the deformable membrane 20 to the home state. Thereafter, the venting valve 16 is opened to permit the pressure inside the chamber 100 to return to the atmospheric pressure through the venting port 17. Please note that the pressure inside the chamber 100 is increased gradually so as to avoid blowing away the test sample (S) from the micropipette 3. In some other embodiments, before reduction to reach the stress pressure (Pₛ), the pressure (Pₕ) generated at the suction port 301 of the micropipette 3 may be kept at a slightly negative pressure (e.g., −0.01±10% psi) for a time period (e.g., 2±10% seconds) and for sticking the test sample (S).

During the testing process shown in FIG. 12, the driving shaft 32 can be driven to move between the proximate and distal positions.

In some embodiments in which the deformable membrane 20 is deformed inwardly in the deformed state (e.g., the embodiment shown in FIGS. 1 and 2), (i) at the time period (tₕ), the driving shaft 32 is in the distal position and the deformable membrane 20 is in the deformed state (see FIG. 2), and (ii) at the time period (tₛ), the driving shaft 32 is in the proximate position, the deformable membrane 20 is in the flat state (see FIG. 1).

In some other embodiments in which the deformable membrane 20 is deformed outward in the deformed state (e.g., the embodiment shown in FIGS. 3 and 4), (i) at the time period (tₕ), the driving shaft 32 is in the distal position and the deformable membrane 20 is in the flat state (see FIG. 3), and (ii) at the time period (tₛ), the driving shaft 32 is in the proximate position and the deformable membrane 20 is in the deformed state (see FIG. 4).

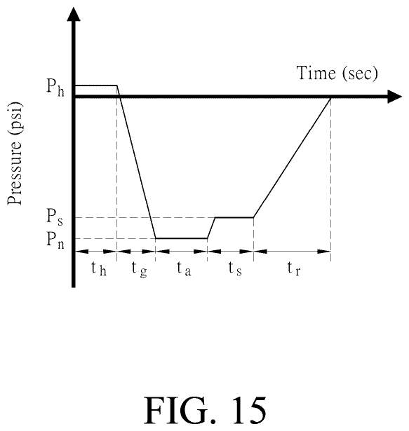

FIG. 15 is a graph illustrating another testing process for determining the quality of the test sample (S) in accordance with some embodiments. The testing process shown in FIG. 15 is described with reference to FIGS. 10, 13 and 14. During the testing process illustrated in FIG. 15, the adjusting valve 15 is switched to adjust the pressure generated at the suction port 301 of the micropipette 3.

Referring to FIGS. 10 and 15, in the beginning, the venting valve 16 is fully closed, the adjusting valve 15 is fully closed, and the pressure inside the chamber 100 is adjusted to and kept at an initial pressure (e.g., a lower positive pressure), and the position of the driving shaft 32 is also moved to permit the deformable membrane 20 to be kept at a home state. For example, in the case that the deformable membrane 20 is deformed inwardly (e.g., the embodiment shown in FIGS. 1 and 2), the home state of the deformable membrane 20 is the deformed state (see FIG. 2); while in the case that the deformable membrane 20 is deformed outwardly (e.g., the embodiment shown in FIGS. 3 and 4), the home state of the deformable membrane 20 is the flat state (see FIG. 3). Due to the initial pressure of the chamber 100, by opening the adjusting valve 15, a slightly positive pressure (Pₕ) (e.g., about 0.01±10% psi) is generated at the suction port 301 of the micropipette 3 and kept for a time period (tₕ) (see FIG. 15), and thus the test sample (S) is retained by the suction port 301 of the micropipette 3 and is prevented from being sucked into the micropipette 3 (see FIG. 13). Then, the adjusting valve 15 is closed, and by actuating the driving device 30 to cause transformation of the deformable membrane 20, a pressure inside the chamber 100 is continuously reduced for a time period (t_{g}) (e.g., 3±10% seconds) so as to reach a preset suction pressure (Pₙ) (e.g., −0.13±10% psi). The preset suction pressure (Pₙ) is kept for a time period (tₐ) (e.g., 2±10% seconds). Then, during a time period (tₛ) (e.g., 4±10% second), by slightly or fully opening the adjusting valve 15, the pressure inside the chamber 100 is slightly increased to and kept at a stress pressure (Pₛ) (e.g., −0.1±10% psi) and in meanwhile, the suction pressure generated at the suction port 301 of the micropipette 3 is substantially equal to the stress pressure (Pₛ). At this stage (i.e., the test sample (S) under the stress pressure (Pₛ)), as shown in a microscope image in FIG. 14, an aspiration depth (D) of the test sample (S) is determined using the computer program (not shown). Finally, the driving device 30 is further actuated to continuously increase the pressure inside the chamber 100 for a time period (tᵣ), thereby returning the deformable membrane 20 to the home state. Thereafter, the venting valve 16 is opened to permit the pressure inside the chamber 100 to return to the atmospheric pressure through the venting port 17. Please note that the pressure inside the chamber 100 is increased gradually so as to avoid blowing away the test sample (S) from the micropipette 3. In some other embodiments, before reduction to reach the preset suction pressure (Pₙ), the pressure (Pₕ) generated at the suction port 301 of the micropipette 3 may be kept at a slightly negative pressure (e.g., −0.01±10% psi) for a time period (e.g., 2±10% seconds) and for sticking the test sample (S).

During the testing process shown in FIG. 15, the driving shaft 32 can be driven to move between the proximate and distal positions.

In some embodiments in which the deformable membrane 20 is deformed inwardly in the deformed state (e.g., the embodiment shown in FIGS. 1 and 2), (i) at the time period (tₕ), the driving shaft 32 is in the distal position and the deformable membrane 20 is in the deformed state (see FIG. 2), and (ii) at the time periods (tₐ, tₛ), the driving shaft 32 is in the proximate position and the deformable membrane 20 is in the flat state (see FIG. 1).

In some other embodiments in which the deformable membrane 20 is deformed outwardly in the deformed state (e.g., the embodiment shown in FIGS. 3 and 4), (i) at the time period (tₕ), the driving shaft 32 is in the distal position and the deformable membrane 20 is in the flat state (see FIG. 3), and (ii) at the time periods (tₐ, tₛ), the driving shaft 32 is in the proximate position and the deformable membrane 20 is in the deformed state (see FIG. 4).

With the provision of the detecting system 2 including the pressure generating device 1, a suction pressure can be generated at the suction port 301 of the micropipette 3 for sucking the test sample (S). Based on the aspiration depth (D) of the test sample (S), the quality of the test sample (S) may be determined. For example, a test sample (S) with a relatively smaller aspiration depth (D) may have a better quality. In addition, because the suction pressure generated using the pressure generating device 1 is relatively small, the test sample (S) is less likely to be damaged during the testing process, and the qualified test sample (S) can be transferred back to the mother for establishing a successful pregnancy.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A pressure generating device, comprising:
a tank having a chamber therein, and including an opening and a communication port each of which is in fluid communication with said chamber;
a deformable membrane which is disposed to seal said opening, and which is deformable between a flat state and a deformed state;
a driving device which is mounted to said tank and which includes a motor body and a driving shaft, said driving shaft having a first shaft end that is disposed to confront said deformable membrane, said driving shaft being driven by said motor body to move between a distal position, where said first shaft end is distal from said motor body, and a proximate position, where said first shaft end is proximate to said motor body; and
a connection unit configured to couple said second shaft end with said deformable membrane and to permit said deformable membrane to be driven by said driving shaft to transform between the flat state, where said first shaft end is in one of the distal portion and the proximate portion, and the deformed state, where said first shaft end is in the other one of the distal position and the proximate position, such that a predetermined pressure is generated through said communication port when said deformable membrane is moved from one of the flat state and the deformed state to the other one of the flat state and the deformed state.

2. The pressure generating device as claimed in claim 1, wherein said deformable membrane is deformed inwardly in the deformed state.

3. The pressure generating device as claimed in claim 1, wherein said deformable membrane is deformed outwardly in the deformed state.

4. The pressure generating device as claimed in claim 1, wherein:
said tank includes
a main casing having said chamber therein, and including said opening and said communication port, and
a side casing detachably mounted to said main casing such that said deformable membrane is secured between said main casing and said side casing to thereby seal said opening;
said motor body is immovably mounted to said side casing; and
said driving shaft extends along a shaft axis, and is driven by said motor body to move toward or away from said deformable membrane so as to transform said deformable membrane between the flat state and the deformed state.

5. The pressure generating device as claimed in claim 4, wherein said main casing extends along a longitudinal axis to terminate at a closed end and a connection end formed with said opening.

6. The pressure generating device as claimed in claim 4, wherein:
said side casing includes
a side casing body that has a distal end and a proximal end opposite to said distal end, and that is formed with a passage extending from said distal end to said proximal end, and
a flange body that extends radially and outwardly from said proximate end of said side casing body, and that is detachably mounted to said main casing; and
said motor body has
an inner portion received in said passage of said side casing body,
an outer portion extending from said inner portion in a direction away from said main casing, and disposed outside said side casing body, and
a flange portion extending radially from said outer portion, and detachably secured to said distal end of said side casing body.

7. The pressure generating device as claimed in claim 1, wherein said connection unit includes:
a connection sleeve which is coupled on said first shaft end of said driving shaft so as to permit said connection sleeve to move with said driving shaft, said connection sleeve having a first sleeve portion, a second sleeve portion having an outer dimension smaller than an outer dimension of said first sleeve portion, and a shoulder surface between said first sleeve portion and said second sleeve portion;
a bearing sleeve which is fittingly sleeved on said second sleeve portion so as to permit said bearing sleeve to move with said connection sleeve, and which is disposed between said connection sleeve and said deformable membrane, said bearing sleeve having a first abutment end that is in abutting engagement with said shoulder surface, and a second abutment end that is opposite to said first abutment end; and
a bearing cap having
an engaging portion formed with a recess that is configured to engage with said second abutment end so as to permit said bearing cap to move with said bearing sleeve, and
a mounted portion which is opposite to said engaging portion, and which is secured to a first surface of said deformable membrane so as to transmit a force from said driving shaft to said deformable membrane.

8. The pressure generating device as claimed in claim 7, wherein said deformable membrane is formed with a through hole, and said connection unit further includes a fastener having
an enlarged head which is disposed on a second surface of said deformable membrane opposite to said first surface, and
a fastening rod extending from said enlarged head through said through hole of said deformable membrane to terminate at a rod end, said rod end being fittingly engaged within a hole formed in said mounted portion of said bearing cap so as to permit said deformable membrane to be fastened to said bearing cap through said fastener.

9. The pressure generating device as claimed in claim 7, wherein said connection unit further includes
a first washer disposed between said protruded portion and said first surface of said deformable membrane, and
a second washer disposed between said enlarged head and said second surface of said deformable membrane.

10. The pressure generating device as claimed in claim 1, further comprising a photo sensor which is immovable relative to said tank so as to permit said photo sensor to detect a displacement of said driving shaft.

11. The pressure generating device as claimed in claim 10, further comprising a flag piece which is mounted on a second shaft end of said driving shaft opposite to said first shaft end so as to permit said flag piece to move with said driving shaft, and which has a plurality of detectable positions to be detected by said photo sensor, thereby determining the displacement of said driving shaft.

12. The pressure generating device as claimed in claim 1, wherein
a hardness of said deformable membrane ranges from 0 Shore A to 100 Shore A,
a thrust of said driving device ranges from 100 g to 1000 g, and
a stroke of said driving device ranges from 3 mm to 50 mm.

13. A detecting system for detecting quality of a test sample including an oocyte or an embryo, comprising:
said pressure generating device as claimed in claim 1;
a micropipette for sucking the test sample; and
a connecting tube disposed to connect said communication port of said tank to said micropipette for applying the predetermined pressure to the test sample.

14. The detecting system as claimed in claim 13, wherein an inner diameter of said micropipette ranges from 10 microns to 100 microns.

15. The detecting system as claimed in claim 13, wherein a pressure ranging from 0.5 psi to −0.5 psi is generated at a suction port of the micropipette.

16. The detecting system as claimed in claim 15, wherein the connecting tube includes a first tube segment connected to the communication port of the tank, and a second tube segment connected to the micropipette, said first and second tube segments being connected to each other through an adjusting valve for adjusting the pressure generated at said suction port of the micropipette.
